(19) United States
(12) Patent Application Publication
(10) Pub. No.: US 2011/0013124 A1
(43) Pub. Date: Jan. 20, 2011

(54) LYOTROPIC CHROMOPHORIC COMPOUNDS, LIQUID CRYSTAL SYSTEMS AND OPTICALLY ANISOTROPIC FILMS

(75) Inventors: Shuangxi Wang, Corona, CA (US); Zongcheng Jiang, Oceanside, CA (US); Michiharu Yamamoto, Carlsbad, CA (US)

Correspondence Address: KNOBBE MARTENS OLSON & BEAR LLP, 2040 MAIN STREET, FOURTEENTH FLOOR, IRVINE, CA, 92614, US

(73) Assignee: Nitto Denko Corporation, Ibaraki, Osaka (JP)

(21) Appl. No.: 12/921,385

(22) PCT Filed: Mar. 5, 2009
(86) PCT No.: PCT/US09/36163

§ 371 Date: Sep. 7, 2010

Related U.S. Application Data
(60) Provisional application No. 61/034,906, filed on Mar. 7, 2008.

(51) Int. Cl. G02F 1/1335; G02F 1/1337; C07D 221/06; C07D 221/18; C09K 19/34; B05D 5/06

(52) U.S. Cl. 349/96; 349/129; 546/98; 546/38; 546/26; 252/299.61; 427/66

(57) ABSTRACT

Lyotropic chromophoric compounds comprised of a naphthalimide derivative, a perylene-3,4-dicarboxylic imide derivative, or a perylenetetracarboxylic diimide derivative are described. The compounds can be used to form liquid crystal systems possessing high quality optical properties. The resulting liquid crystal systems are readily applied onto a substrate to obtain optically isotropic or anisotropic, at least partially crystalline films applicable in various fields.

### CROSS-REFERENCE TO RELATED APPLICATIONS

This application claims priority to U.S. Provisional Patent Application No. 61/034,906, filed on Mar. 7, 2008, entitled “Lyotropic Liquid Crystal Based Anisotropic Films and Methods of Making.”

### BACKGROUND OF THE INVENTION

1. Field of the Invention

The present invention relates generally to the fields of organic chemistry and optically anisotropic coatings. More specifically, the present invention relates to lyotropic chromophoric compounds, lyotropic liquid crystal systems comprising one or more lyotropic chromophoric compounds, and optically isotropic or anisotropic films.

2. Description of the Related Art

Optical elements are increasingly based on new materials possessing specific, precisely controllable properties. An important element in modern visual display systems is an optically anisotropic film having a combination of optical and other characteristics that can be optimized to suit the requirements of a particular device, since each device often has its own set of requirements.

Various polymeric materials have been used in the manufacture of optically anisotropic films. Films based on such materials may acquire anisotropic optical properties through uniaxial extension and modification with organic dyes or iodine. In many applications, the base polymer is polyvinyl alcohol (PVA). Such films are described in greater detail in the monograph Liquid Crystals: Applications and Uses, B. Bahadur (ed.), World Scientific, Singapore—N.Y. (1990), Vol. 1, p. 101. However, the low thermal stability of PVA-based films can limit their application. Development of new materials and methods for the synthesis of optically anisotropic films possessing improved characteristics is therefore quite advantageous. Particularly, films having properties such as higher heat resistance, convenient synthesis, and uniformity are highly desirable.

Organic dichroic dyes have gained prominence in the manufacture of optically anisotropic films with improved optical and working characteristics. Films based on these compounds may be obtained by applying a layer of a liquid crystal (LC) aqueous dye solution containing dye supramolecules onto a substrate surface followed by evaporation of the solvent. The resulting LC films acquire can anisotropic properties in several ways. For example, the anisotropic properties can be acquired by preliminary mechanical ordering of the underlying substrate surface as described, for example, in U.S. Pat. No. 2,553,961. Or, the anisotropic properties can be acquired by subsequent application of external mechanical, electromagnetic, or other orienting forces to the LC coating on the substrate as described, for example, in PCT Publication No. WO 94/28073.

Investigations into the application of LC dyes, as well as the properties of related systems have become more extensive in the past fifteen years. Recent studies into these phenomena have been motivated largely by industrial applications in liquid crystal displays (LCD's) and glazing. The dye supramolecules may form lyotropic liquid crystal (LLC) phases in which the dye molecules pack into supramolecular complexes that are generally shaped like columns, which are the basic structural units of a mesophase. A high degree of ordering of dye molecules in the columns allows such mesophases to be used for obtaining oriented films characterized by a strong dichroism.

Dye molecules that form supramolecular LC mesophases typically include peripheral groups that render the dyes water soluble. The mesophases of organic dyes are characterized by specific structures, phase diagrams, optical properties, and dissolving capabilities, as described for example in J. Lydon, Chromonics, Handbook of Liquid Crystals (Wiley—VCH, Weinheim, 1998), Vol. 2B, pp. 981 to 1007.

Previous research has also focused on thermotropic LC compounds. While thermotropic LC compounds may be oriented into anisotropic films by mechanical forces, such orientation may disappear when the mechanical forces are discontinued. In contrast, LLC phases often retain their dichroic orientation even when a mechanical force is applied and then removed.

Such properties of LLC phases account for the growing interest in LLC materials, prompting the development of methods for preparing films based on organic dyes. Recent improvements have involved both film application conditions and identification of new LLC systems. In particular, new LLC compositions for the synthesis of optically anisotropic films may be obtained by introducing modifiers, stabilizers, surfactants, and other additives to known dyes as described in, for example, published PCT Publication No. WO 94/28073.

Recently, there has been increasing demand for films possessing high optical anisotropy that are also characterized by improved selectivity in various wavelength ranges. Films whose absorption maxima occur at different locations in the wide spectral range from the infrared (IR) to the ultraviolet (UV) are very desirable. Several compounds have been developed that are capable of forming LLC films possessing these characteristics. However, the number of dyes known to form stable lyotropic mesophases remains relatively small.

Disulfoderivative organic dyes, including perylenetetracarboxylic acid (PTCA) based compounds, are important water-soluble dichroic dyes capable of forming stable LLC phases. PCTA species applicable in the manufacturing of optically anisotropic films are described in PCT Publication No. WO 94/28073 and U.S. Pat. Nos. 7,025,900 and 7,160,485. In general, PTCA derivatives are characterized by excellent chemical, thermal, and photochemical stability.

To improve the solubility of the perylene dyes in organic solvents, various substituents have been introduced into the molecules. Examples of such substituents include oxyethyl groups as described in Cormier et al., Phys. Chem. 101 (51), 11004 to 11006 (1997) and phenoxy groups as described in Quante et al., Chem. Mater. 6(2), 495 to 500 (1997). Solubility of perylene dyes may also be increased by substitution with amino groups, as described in Iverson, et al., Langmuir 18(9), 3510 to 5316 (2002), and by substitution with sulfonic groups, as described in PCT Publication No. WO 94/28073 and U.S. Pat. No. 7,025,900. Increased solubility may also be obtained through the substitution of carboxylic groups. Various dye compositions (also referred to as “inks”) used in the manufacture of polarizer films based on other PTCA sulfoderivatives have been disclosed in U.S. Pat. No. 5,739,296, U.S. Pat. No. 7,160,485, Japanese Pat. App. 2006-098927, and U.S. Pat. App. Pub. No. 2006/0272546

Optically anisotropic films may be formed on glass, plastic, or other substrate materials. Films which exhibit high quality optical characteristics, such as those films having dichroic ratios that approach the range of approximately 25 to 30, may be used as polarizers, which are described in Bobrov, et al., Environmental and Optical Testing of Optiva Thin Crystal Film® Polarizers, Proceedings of the 10th SID Symposium “Advanced display technologies,” (Minsk, Republic of Belarus, Sep. 18-21, 2001), p. 23 to 30. Methods for the preparation of such films, including those with a high degree of crystallinity, are described in PCT Publication No. WO 02/063,660. The aforementioned PTCA derivatives are capable of forming LLC phases, and anisotropic films obtained using the LLC system possess excellent optical characteristics and exhibit good performance as polarizers.

One disadvantage of manufacturing anisotropic films is that obtaining reproducible samples can be difficult. Currently, film application technologies generally require that the process parameters, such as concentration of the reactants, temperature for the film formation, etc., be carefully selected and strictly maintained. However, even if all of the processing conditions used in film formation are precisely followed, random local variation of the coating regime can still occur. This may cause the formation of misorientation zones and microdefects as a result of non-uniform micro- and macro-crystallization processes in the course of solvent removal. In addition, the manufacture of LLC systems carries the risk of non-uniform thickness of the applied coating, which also decreases reproducibility of the film parameters.

Accordingly, it is desirable to develop different compounds and/or different methods of film application/formation that can provide reproducible LLC films and systems having good optical characteristics. Each of the references cited above is hereby incorporated by reference in their entirety, particularly for the purpose of describing manufacturing methods of the optical compounds, LLC systems, and device applications.

### SUMMARY OF THE INVENTION

An embodiment provides a lyotropic chromophoric compound. In an embodiment, the lyotropic chromophoric compound comprises a naphthalimide derivative. In an embodiment, the lyotropic chromophoric compound comprises a perylene-3,4-dicarboxylic imide derivative. In an embodiment, the lyotropic chromophoric compound comprises a perylenetetracarboxylic diimide derivative. In an embodiment, the lyotropic chromophoric compound is a compound having the general structural formula (I), a compound having the general structural formula (II), or a compound having the general structural formula (III):

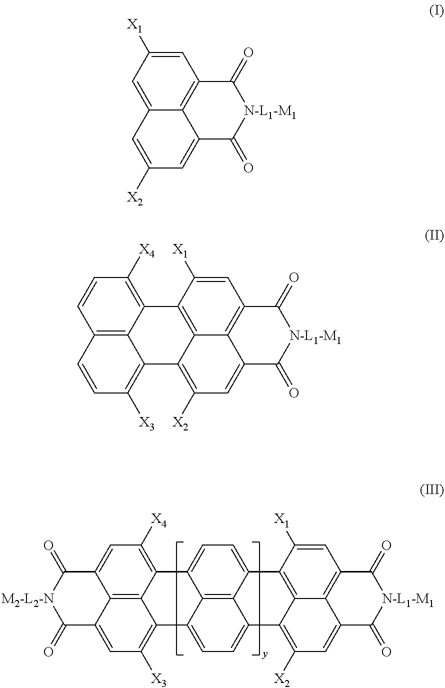

wherein L₁ and L₂ each independently represent a hydrophilic linker; M₁ and M₂ each independently represent an acidic group, a basic group, or salt thereof; X₁, X₂, X₃ and X₄ are each independently selected from —H, —NHCH₃, a pyrrolidinyl group, or a halogen; and y is an integer in the range from 0 to about 4.

The lyotropic chromophoric compounds described herein can be used in optical devices and systems used to manufacture such devices. An embodiment provides a lyotropic liquid crystal system comprising at least one lyotropic chromophoric compound as described above. In an embodiment, the lyotropic liquid crystal system comprises a solvent, such as water or water intermixed with an organic solvent. The compounds described herein can be used in the manufacture of anisotropic or isotropic optical films. Another embodiment provides an optically anisotropic film comprising at least one lyotropic chromophoric compound as described herein. The film can be formed by applying a lyotropic liquid crystal system described herein onto a substrate. The films described herein can be used in the manufacture of liquid crystal display devices. In an embodiment, the film has a dichroic ratio greater than or equal to about 20. In an embodiment, the film has a dichroic ratio greater than or equal to about 25. In an embodiment, the film has a dichroic ratio greater than or equal to about 30.

These and other embodiments are described in greater detail below.

### BRIEF DESCRIPTION OF THE DRAWINGS

FIG. 1 is a synthetic scheme showing one embodiment providing a method of synthesizing a sulfoderivative of perylenedicarboxylic imide.

FIG. 2 is a synthetic scheme showing one embodiment providing a method of synthesizing a sulfoderivative of perylenetetracarboxylic diimide.

FIG. 3 is a synthetic scheme showing one embodiment providing a method of synthesizing a pyridinium derivative of perylenetetracarboxylic diimide.

### DETAILED DESCRIPTION OF THE PREFERRED EMBODIMENT

Described herein are lyotropic chromophoric compounds that are capable of forming stable liquid crystals, and methods of synthesizing such compounds. The lyotropic chromophoric compounds described herein may generally be referred to as chromophores. Also provided are LLC systems, comprising a solvent and one or more lyotropic chromophoric compounds as described herein. Also provided are isotropic, anisotropic, or at least partially crystalline films based on these systems and compounds, and methods for manufacturing such films. Embodiments of the films described herein possess excellent optical properties and working characteristics.

Using dichroic dyes capable of forming LLC systems, it is possible to obtain films possessing a high degree of optical anisotropy. Optically anisotropic films may be formed on glass, plastic, or other substrate materials. Because they exhibit high quality optical characteristics and have dichroic ratios that are greater than 25, e.g., in the range of about 25 to about 130, these films may be used as polarizers. Such films exhibit the properties of E-type polarizers, which are related to peculiarities of the optical absorption of supramolecular complexes, and behave as retarders (i.e., phase-shifting devices) in the spectral regions where the absorption is insignificant. The phase-retarding properties of these anisotropic films are related to their birefringence, that is, a difference in the refractive indices measured in the direction of application of the LLC system onto a substrate and in the perpendicular direction. A preferred LLC film formed from a strong (preferably light-fast) dye molecule-based LLC system is characterized by a high thermal stability and a good resistance to fading.

These and other advantages of the embodiments described herein can be achieved with a lyotropic chromophoric compound comprising a naphthalimide derivative having the general structural formula (I), a perylene-3,4-dicarboxylic imide derivative having the general structural formula (II), or a perylenetetracarboxylic diimide derivative having the general structural formula (III), described above.

Each of the hydrophilic linking groups L₁ and L₂ in formulae (I), (II), and (III) can be independently selected. L₁ and L₂ can be the same or different. A “hydrophilic linker” as described herein is a linking group with a length and composition that is effective to render the compound to which they are attached sufficiently soluble, such that the compound can react with a counter ion in a suitable solvent such as water. The hydrophilic linker need not, however, render the compound completely soluble in the chosen solvent before the counter ion is added. However, the hydrophilic linker should render the compound soluble in the solvent once a salt is formed with the counter ion. In an embodiment, the compound is at least partially soluble in water. In an embodiment, the compound is soluble in water. Preferably, L₁ and L₂ in formulae (I), (II), and (III) are each independently selected from a polyethyleneglycol linker having the general formula (IV), a polypropyleneglycol linker having the general formula (V) and a polyethyleneimine linker having the general formula (VI):

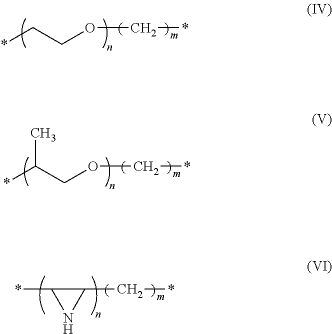

wherein each n in formulae (IV), (V), and (VI) is independently selected from an integer in the range of 1 to about 9 and each m is independently selected from an integer in the range of 0 to about 6. In an embodiment, each n in formulae (IV), (V), and (VI) is selected from an integer in the range of 1 to about 8. In an embodiment, each n in formulae (IV), (V), and (VI) is selected from an integer in the range of 1 to about 4. In an embodiment, each n in formulae (IV), (V), and (VI) is selected from an integer in the range of 2 to about 5. In an embodiment, each n in formulae (IV), (V), and (VI) is selected from an integer in the range of 3 to about 6. As n is increased, the hydrophilic nature of the hydrophilic linker is also increased.

M₁ and M₂ in formulae (I), (II), and (III) each independently represent an acidic group, a basic group, or salt thereof. M₁ and M₂ can be the same or different. In an embodiment, the acidic group, basic group, or salt thereof comprises nitrogen. In an embodiment, the acidic group, basic group, or salt thereof comprises sulfur. In embodiments where M₁ and/or M₂ of the chromophoric compound comprise an acidic group, the acidic group can be converted to a salt by intermixing the chromophoric compound with a suitable base. In embodiments where M₁ and/or M₂ of the chromophoric compound comprise a basic group, the basic group can be converted to a salt by intermixing the chromophoric compound with an acid. Selection of the counter ion, e.g. formed from the reaction with the acid or base, can be determined by those having ordinary skill in the art, guided by the disclosure herein. Each M₁ and M₂ can be selected to be salts that configure the compound to be soluble in water or water intermixed with another organic solvent. For example, conversion of the acidic or basic groups into salts can increase the solubility of the compound. Thus solubility of the compound can be controlled by selection of the hydrophilic linker, e.g. length of the hydrophilic portion of the hydrophilic linker, and the salt group of M₁ and/or M₂.

In an embodiment, M₁ and M₂ are each independently selected to comprise an anion portion independently selected from —SO₃⁻ and —CO₂⁻. The anion portion of M₁ and M₂ that is covalently attached the compound can be ionically bonded to one or more counter ions. In an embodiment, each M₁ and M₂ further comprises one or more counter ion. In an embodiment, the counter ion is independently selected from H⁺, NH₄⁺, K⁺, Li⁺, Na⁺, Cs⁺, Ca⁺⁺, Sr⁺⁺, Mg⁺⁺, Ba⁺⁺, Co⁺⁺, Mn⁺⁺, Zn⁺⁺, Cu⁺⁺, Pb⁺⁺, Fe⁺⁺, Ni⁺⁺, Al³⁺, Ce³⁺, La³⁺, or a protonated organic amine, or similar counter ions. Examples of suitable protonated organic amines include NH(Et)₃⁺, NH₂(Et)₂⁺, NH₃(Et)⁺, NH(Me)₃⁺, NH₂(Me)₂⁺, NH₃(Me)⁺, H₃NCH₂CH₂OH⁺, and H₂NCH₂(CH₂OCH₂CH₂OH)⁺. In an embodiment, the counter ion is independently selected from NH₄⁺ and NH(Et)₃⁺. The number of counter ions can vary and may be fractional if the counter ion or ions are associated with more than one molecule. In an embodiment, one or more counter ions are shared by at least two molecules.

In an embodiment, M₁ and M₂ are each independently selected to comprise a cation portion independently selected from:

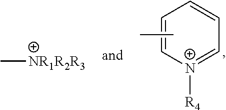

wherein R₁, R₂, R₃, and R₄ are each independently selected from hydrogen, an optionally substituted C₁ to C₆ alkyl group, an optionally substituted C₂ to C₆ alkenyl group, an optionally substituted C₂ to C₆ alkynyl group, an optionally substituted C₃ to C₈ cycloalkyl group, an optionally substituted aryl group, or an optionally substituted aralkyl group. An appropriate counter ion can be selected. In an embodiment, the counter ion is independently selected from CO₂CF₃⁻, CH₃SO₃⁻, Cl⁻, Br⁻, and I⁻. In an embodiment, the counter ion is CH₃SO₃⁻. The number of counter ions can vary and may be fractional if the counter ion or ions belong to more than one molecule. In an embodiment, one or more counter ions are shared by at least two molecules.

Each of the alkyl, alkenyl, alkynyl, cycloalkyl or aryl groups in R₁, R₂, R₃, and R₄ as described above can be “optionally substituted” with one or more substituent group(s). When substituted, the substituent group(s) is (are) one or more group(s) individually and independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. Non-limiting examples of the substituent group(s) include methyl, ethyl, propyl, butyl, pentyl, isopropyl, methoxide, ethoxide, propoxide, isopropoxide, butoxide, pentoxide and phenyl.

The alkyl, alkenyl, and alkynyl groups in R₁, R₂, R₃, and R₄ can be linear or branched groups. Some examples of R₁, R₂, R₃, and R₄ as alkyl groups include methyl, ethyl, n-propyl, isopropyl, n-butyl, isobutyl, sec-butyl, and tert-butyl. Additionally, R₁, R₂, R₃, and R₄ can be various cycloalkyl groups. For example, the cycloalkyl group can include cyclopentyl, cyclohexyl, or cyloheptyl. Some examples of useful aryl groups include phenyl, tolyl, naphthyl, phenanthryl, and anthracenyl. Some examples of useful aralkyl groups include benzyl, phenethyl, naphthylmethyl, phenanthylmethyl, and anthranylmethyl. Preferably, R₁, R₂, R₃, and R₄ are independently selected from hydrogen, methyl, ethyl, n-propyl, isopropyl, butyl, t-butyl, and cyclohexyl.

X₁, X₂, X₃ and X₄ in formulae (I), (II), and (III) are each independently selected from —H, —NHCH₃, a pyrrolidinyl group, or a halogen. In an embodiment, the halogen can be selected from fluorine, chlorine, iodine, or bromine. In an embodiment, X₁, X₂, X₃ and X₄ are each selected to be hydrogen. In an embodiment, X₁ and X₂ in formula (I) are selected to be different substituents. In an embodiment, at least one of X₁, X₂, X₃ and X₄ in formulae (I), (II), and (III) is selected to be different from the other substituents.

In an embodiment, y in formulae (I), (II), and (III) is selected to be an integer in the range of 0 to 4. As y is increased, the aromatic nature of the compound is also increased. For example, the absorbance peak of the compound can be shifted to longer wavelengths with increased aromaticity. This allows for absorbance peaks at various visible colors. Increasing aromaticity can also decrease the solubility of the compound. In an embodiment, y is selected to be an integer in the range of 0 to about 2. The m in formulae (IV), (V), and (VI) is selected to control the distance between the hydrophilic portion of the hydrophilic linker and the acidic group, basic group, or salt thereof. In an embodiment, m is selected to be an integer in the range of 1 to about 3.

In an embodiment, the compounds described herein are configured for π-π stacking. The aromatic groups present in the compound can allow for two-dimensional π-π stacking.

An “LLC system” as described herein is a solution comprising a solvent and one or more lyotropic chromophoric compounds as described herein. In an embodiment, the LLC system comprises an LLC mesophase. An LLC mesophase is formed when the concentration of lyotropic chromophoric compound in an LLC system is at or above the critical concentration for the formation of a liquid crystal within the system. The compounds described herein can be configured to absorb light in the visible spectrum range and also can be configured to form LLC systems with increased stability over thermotropic liquid crystals. These stable LLC systems may be used in the formation of anisotropic, isotropic, and/or at least partially crystalline films with highly reproducible, optimal optical characteristics. Film formation with greater uniformity and fewer microdefects upon solvent removal can be accomplished using embodiments of the LLC systems comprising the lyotropic chromophoric compounds described herein.

Embodiments of the LLC systems formed with the compounds described herein further possess increased stability over a broad range of concentrations, temperatures, and pH ranges. Thus, the systems and compounds simplify the process of anisotropic film formation and permit the use of a variety of techniques for creation of film layers. The production of films is facilitated with highly reproducible parameters. Embodiments of the organic compounds described herein exhibit improved aqueous solubility. The increased optical anisotropy demonstrated by embodiments of the films comprising the chromophoric compounds is highly desirable. Without being bound by theory, the inventors believe that the high degree of optical anisotropy exhibited by certain embodiments is derived through non-covalent bonding, such as hydrogen bonding and cation-anion interactions, between two or more molecules.

The LLC systems can be formed over a broad range of pH. For example, the acidic, basic, or salt characteristic of M₁ and M₂ can be adjusted by one of ordinary skill in the art to affect the solubility in various pH solutions. In an embodiment, M₁ and/or M₂ comprises an acidic group, which the compound has a pH in the range of about 1 to about 6 in solution, depending on the concentration of the compound. In an embodiment, M₁ and/or M₂ comprises a basic group, which the compound has a pH in the range of about 8 to about 12 in solution, depending on the concentration of the compound.

Conversion of the acidic or basic groups into their salt forms can also be used to adjust the solubility of the compound. For example, solubility in water can further be controlled by selection of the appropriate counter ion. Additionally, certain counter ions, such as Li⁺ among others, can improve the dichroic ratio of the compound.

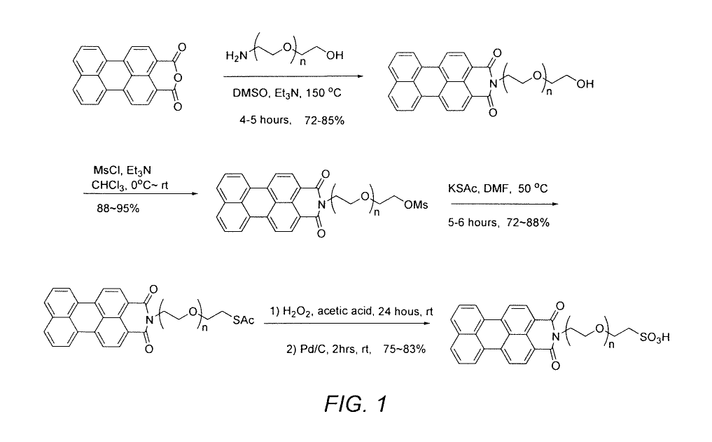

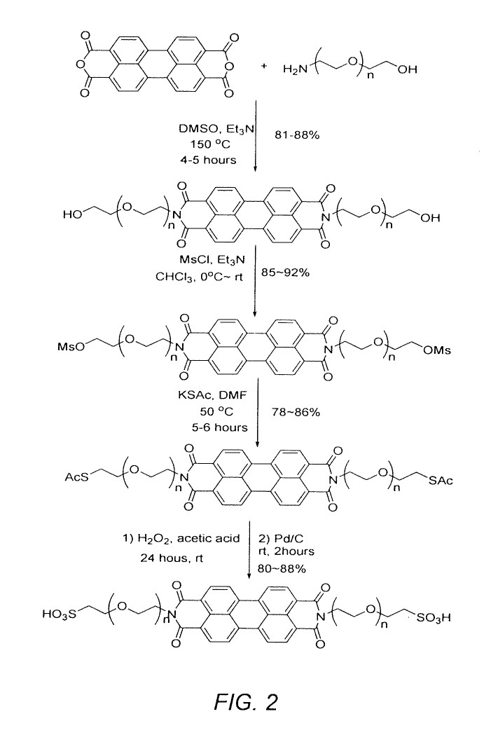

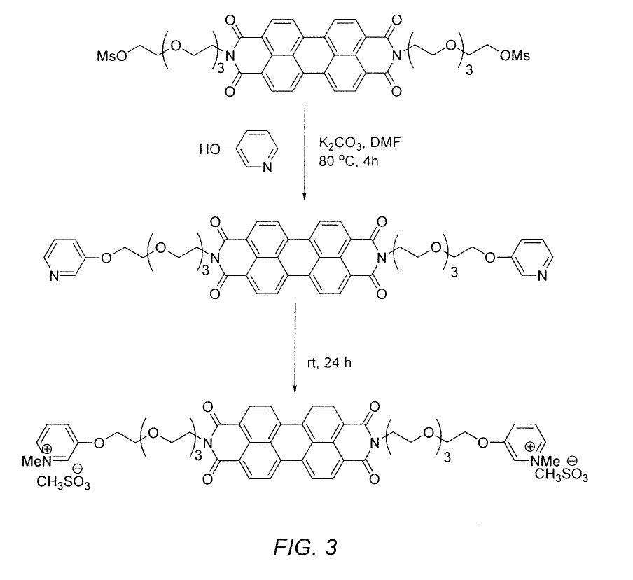

The compounds described herein can be synthesized by one having ordinary skill in the art, guided by the disclosure herein, by way of commonly used techniques used to synthesize analogous lyotropic organic structures. For example, as shown in FIGS. 1 and 2, controlled amounts of perylenedicarboxylic monoanhydride or perperylenetetracarboxylic dianhydride are reacted with amino-polyethylene oxide-ethanol [NH₂—(CH₂CH₂O)ₙ—CH₂CH₂OH] for 4-5 hours at 150° C. using anhydrous trimethyl amine as a base and dimethyl sulfoxide (DMSO) as a solvent under argon. The resulting product is further reacted with methanesulfonyl chloride in anhydrous dichloromethane in presence of triethylamine at 0° C., followed by reaction with potassium thioacetate in DMF for 5-6 hours. The dark red colored product is finally oxidized by hydrogen peroxide in acetic acid to form the resulting dye, a water-soluble sulfonated perylene-3,4-dicarboxylic imide or perylenetetracarboxylic diimide derivative with side chains of variable polyethylene oxide lengths, depending on the n in the amino-polyethylene oxide ethanol. A method of making perylenetetracarboxylic diimide pyridinium derivatives is shown in FIG. 3. Polarized microscopic analysis of the system texture reveals that a stable lyotropic mesophase can be formed at room temperature at a dye concentration of about 5% to about 30% by wt. Accordingly, a nematic phase is observed within a sufficiently narrow range of dye concentrations and temperatures. The existence of isotropic phases and their boundaries, as well as two-phase transition regions, may be readily determined in this system.

The compounds having the general structural formulae (I), (II), or (III) can form stable LLC systems both individually and in mixtures. Various combination of compounds of formulae (I), (II), and (III) can be used in the manufacture of LLC systems and films. Furthermore, each of these compounds can be mixed with other known lyotropic compounds.

In an embodiment, the compounds having the general structural formulae (I), (II), and/or (III) are combined with other dichroic dyes capable of forming LLC phases to form LLC systems. In an embodiment, the compounds having the general structural formulae (I), (II), and/or (III) are combined with other substances that are generally non-absorbing (colorless) or weakly absorbing in the visible range and capable of forming LLC systems. The LLC systems can be formed, for example, by intermixing the compounds with a solvent, such as water. After removal of the solvent, this LLC system can form an anisotropic, isotropic and/or at least partially crystalline film with reproducibly high optical characteristics. Methods and systems for forming stable LLC systems and resultant anisotropic, isotropic and/or at least partially crystalline optical films are described in greater detail in U.S. Pat. No. 6,563,640, the disclosure of which is incorporated by reference, particularly for the purpose of describing optical films and methods for making them.

Lyotropic chromophoric compounds in aqueous solutions as described herein typically exhibit a maximum optical absorption in the wavelength interval between about 400 nm to about 780 nm. In an embodiment, the chromophoric compounds in aqueous solutions exhibit a maximum optical absorption in the wavelength interval between about 450 nm to about 700 nm. The hydrophilic-hydrophobic balance of the molecular aggregates formed in LLC systems can be controlled when using the compounds described herein. For example, the chromophoric perylene core structure in formula (III) can be adjusted by varying y (to produce tetra perylene or higher orders) to increase hydrophobicity. Furthermore, the length of the polyethyleneglycol linker having the general formula (IV), the polypropyleneglycol linker having the general formula (V), and/or the polyethyleneimine linker having the general formula (VI) can be increased to adjust hydrophilicity. By varying either or both of these parameters, one of ordinary skill can change the solubility of the compound and the solution viscosity when mixed with a solvent. Additionally, one of ordinary skill can also adjust the absorption wavelengths and produce chromophoric compounds that cover all or part of the full color wavelength spectrum.

Embodiments of the lyotropic chromophoric compounds described herein can be used to form stable lyotropic liquid crystal systems. LLC systems of individual compounds having the general structural formulae (I), (II), or (III), as well as mixtures of such compounds, can be prepared by one of ordinary skill in the art, guided by the disclosure herein.

One or more of the compounds described herein can be intermixed with a solvent to form an LLC system, which can then be applied onto a substrate surface and oriented by any known method such as, for example, those described in PCT Publication Nos. WO 94/28073 and WO 00/25155, the disclosures of which are incorporated by reference. The types of substrate suitable for making optically anisotropic films may include transparent/translucent substrates, such as glass, plastic, color filter, and transparent/translucent polymer sheet, and semiconductors. In some embodiments, the LCC system is applied onto a substrate by means of spraying, pouring, printing, coating, dipping or transferring by a spoon, a spatula, a rod or any object capable of transferring a liquid crystal system. The desired orientation of the liquid crystals may be provided, for example, by applying shear stress, gravitational force, or an electromagnetic field. In some embodiments, an applicator rod or suitable tools may be used to apply pressure on the surface to orient or arrange the LLC system. A linear velocity in the range of about 25 mm/s to about 1 m/s can be applied on the film surface to orient the liquid crystal mesophases. The film forming process may be carried out at room temperature. In some embodiments, the relative humidity during orientation may be in the range of from about 55% to about 85%. In some embodiments, diimides described herein provide one of the simple ways to line up the molecules by requiring only a minimal mechanical “spreading” with a glass rod onto the substrate to orient the LLC systems. In an embodiment, the LLC system comprises an LLC mesophase. In one embodiment, the LLC systems are oriented by spreading the LLC system in one direction.

Subsequent removal of the solvent from the oriented liquid crystal solution can be carried out to form an optically anisotropic film with a thickness in the range of about 0.1 μm to about 2 μm. In an embodiment, the film has a thickness in the range of about 0.2 μm to about 1 μm. In an embodiment, the film has a thickness in the range of about 0.3 μm to about 0.5 μm. In some embodiments, the anisotropic film may also be a polycrystalline film.

To improve substrate wetting and optimization of the rheological properties of a liquid crystal system, the solution can be modified, for example, by adding plasticizing water-soluble polymers and/or anionic or non-ionic surfactants. The LLC system may further comprise one or more water-soluble, low-molecular-weight additives. Each of the additives can be advantageously selected so as not to destroy the alignment properties of the liquid crystal system. Examples of water-soluble, low-molecular-weight additives include, but are not limited to, plasticizing polymer, such as PVA and polyethylene glycol, and anionic or non-ionic surfactants such as those available under the tradename TRITON, which is a nonionic surfactant having hydrophilic polyethylene oxide groups and a hydrocarbon lipophilic or hydrophobic group. These additives may improve substrate wetting and optimize the rheological properties of an LLC system. All additives are preferably selected so as not to destroy the alignment properties of the LLC system.

Embodiments of the films formed from the LLC systems described herein can be generally characterized by an approximately 10% or greater performance advantage, e.g., increase in reproducibility of one or more performance parameters from batch to batch, between different films in the same batch, and over the surface of one film as compared to the other films.

The compounds described herein may be also used to obtain isotropic films. For example, the LLC system comprising a compound having the general structural formula (I), (II), or (III) and a solvent may be applied onto a substrate and not be subjected to any external orienting action. This can be achieved through application of the LLC system by methods such as spraying, offset printing, and silk screening. Removal of the solvent leaves the substrate covered with a polycrystalline film with an overall domain structure that possesses isotropic optical properties.

The lyotropic chromophoric compounds can be used to form at least partially crystalline films and/or polarizing films and/or birefringent films. These lyotropic chromophoric compounds may be used in the production of optically isotropic or anisotropic, polarizing films and/or phase-retarding films and/or birefringent films. In an embodiment, the LLC system used to form an optically isotropic or anisotropic film comprises at least two compounds selected from the general structural formulae (I), (II), and (III). In another embodiment, the LLC system is used to form an optically isotropic or anisotropic film comprising at least two specific compounds of at least one of formulae (I), (II), and (III), wherein the two specific compounds comprise at least two different substituents for X₁, X₂, X₃, or X₄. In some embodiments, the LLC system may encompass an aqueous liquid crystal solution that may be referred to as a “water-based ink composition.”

In an embodiment, the LLC system is water-based. For example, the LLC system can comprise one or more compounds of the disclosed lyotropic chromophores having the general structural formulae (I), (II), and/or (III) and water. Other solvents can also be used. In an embodiment, the LLC system comprises a mixture of water and an organic solvent miscible with water. In an embodiment, the LLC system comprises a mixture of water and an organic solvent, which is alternatively miscible with water in any proportion or characterized by limited miscibility with water. Useful organic solvents include polar solvents, such as dimethyl sulfoxide (DMSO), dimethylformamide (DMF), alcohol (e.g., methanol or ethanol) and N-Methyl-2-pyrrolidone (NMP).

Other materials known to those having ordinary skill in the art may also be included. In an embodiment, the LLC system further comprises one or more surfactants. In an embodiment, the surfactant is present in an amount of up to about 5% by weight of the LLC system. In an embodiment, the surfactant is present in an amount in the range of about 0.1% to about 1% by weight of the LLC system. In an embodiment, the LLC system further comprises one or more plasticizers. In an embodiment, the plasticizer is present in an amount of up to about 5% by weight of the LLC system. In an embodiment, the plasticizer is present in an amount in the range of about 0.1% to about 1% by weight of the LLC system.

The concentration of the lyotropic chromophoric compound or mixture of lyotropic chromophoric compounds in the LLC systems described herein can vary. In an embodiment, the concentration of the lyotropic chromophoric compound in the LLC system is in the range of about 5% to about 50% by weight of the LLC system. In an embodiment, the concentration of the lyotropic chromophoric compound in the LLC system is in the range of about 8% to about 40% by weight of the LLC system. In an embodiment, the concentration of the lyotropic chromophoric compound in the LLC system is in the range of about 10% to about 30% by weight of the LLC system.

The concentration of individual lyotropic chromophoric compounds in the LLC system can also vary, depending on the required properties of the film, as described below. In an embodiment, the LLC system comprises a combination of two or more compounds of the general structural formulae (I), (II), and/or (III), wherein the amount of compound according to formula (I) is in the range of about 0% to about 99% by weight, based on the total amount of chromophoric compounds, the amount of compound according to formula (II) is in the range of about 0% to about 99% by weight, based on the total amount of chromophoric compounds, and the amount of compound according to formula (III) is in the range of about 0% to about 99% by weight, based on the total amount of chromophoric compounds. Optionally, the total amount of compounds according formulae (I), (II), and/or (III) can account for at least 50% of the total weight of chromophoric compounds. Optionally, the total amount of compounds according formulae (I), (II), and/or (III) can account for at least 75% of the total weight of chromophoric compounds. Optionally, the total amount of compounds according formulae (I), (II), and/or (III) can account for at least 90% of the total weight of chromophoric compounds. Optionally, the total amount of compounds according formulae (I), (II), and/or (III) can account for about 100% of the total weight of chromophoric compounds.

In an embodiment, the amount of compound according to formula (I) in the LLC system is in the range of about 1% to about 100% by weight, based on the total amount of chromophoric compounds. In an embodiment, the amount of compound according to formula (I) in the LLC system is in the range of about 5% to about 95% by weight, based on the total amount of chromophoric compounds. In an embodiment, the amount of compound according to formula (I) in the LLC system is in the range of about 10% to about 90% by weight, based on the total amount of chromophoric compounds. In an embodiment, the amount of compound according to formula (I) in the LLC system is in the range of about 20% to about 80% by weight, based on the total amount of chromophoric compounds. In an embodiment, the amount of compound according to formula (I) in the LLC system is in the range of about 1% to about 50% by weight, based on the total amount of chromophoric compounds. In an embodiment, the amount of compound according to formula (I) in the LLC system is in the range of about 50% to about 99% by weight, based on the total amount of chromophoric compounds.

In an embodiment, the amount of compound according to formula (II) in the LLC system is in the range of about 1% to about 100% by weight, based on the total amount of chromophoric compounds. In an embodiment, the amount of compound according to formula (II) in the LLC system is in the range of about 5% to about 95% by weight, based on the total amount of chromophoric compounds. In an embodiment, the amount of compound according to formula (II) in the LLC system is in the range of about 10% to about 90% by weight, based on the total amount of chromophoric compounds. In an embodiment, the amount of compound according to formula (II) in the LLC system is in the range of about 20% to about 80% by weight, based on the total amount of chromophoric compounds. In an embodiment, the amount of compound according to formula (II) in the LLC system is in the range of about 1% to about 50% by weight, based on the total amount of chromophoric compounds. In an embodiment, the amount of compound according to formula (II) in the LLC system is in the range of about 50% to about 99% by weight, based on the total amount of chromophoric compounds.

In an embodiment, the amount of compound according to formula (III) in the LLC system is in the range of about 1% to about 100% by weight, based on the total amount of chromophoric compounds. In an embodiment, the amount of compound according to formula (III) in the LLC system is in the range of about 5% to about 95% by weight, based on the total amount of chromophoric compounds. In an embodiment, the amount of compound according to formula (III) in the LLC system is in the range of about 10% to about 90% by weight, based on the total amount of chromophoric compounds. In an embodiment, the amount of compound according to formula (III) in the LLC system is in the range of about 20% to about 80% by weight, based on the total amount of chromophoric compounds. In an embodiment, the amount of compound according to formula (III) in the LLC system is in the range of about 1% to about 50% by weight, based on the total amount of chromophoric compounds. In an embodiment, the amount of compound according to formula (III) in the LLC system is in the range of about 50% to about 99% by weight, based on the total amount of chromophoric compounds.

In an embodiment, a lyotropic liquid crystal system comprises a first compound according to formula (I), (II), or (III), wherein the first compound has a concentration of about 0% to about 50% by mass, and a second compound according to formula (I), (II), or (III) that is different from the first compound, wherein the second compound has a concentration of about 0% to about 50% by mass, wherein the total amount of the first compound and the second compound is up to about 50% by mass, based on the total mass of the LLC system.

In an embodiment, the LLC system further comprises at least one water-soluble organic dye or at least one substantially colorless organic compound. In an embodiment, the organic dye or substantially colorless organic compound is configured to participate in the formation of a liquid crystal. The resulting films can also comprise organic dyes or other organic compounds.

Optically anisotropic films of the present invention may be obtained by applying an LLC system described herein onto a substrate, optionally followed by orienting action, and then drying. Illustrative examples describing the synthesis of lyotropic chromophoric compounds, forming LLC system comprising the compounds, and then forming organic films using the LLC system are described in detail below.

In an embodiment, the optically anisotropic film is formed by depositing an LLC system comprising at least one lyotropic chromophoric compound onto a substrate. In an embodiment, the film is at least partially crystalline. In an embodiment, the film further comprises at least one water soluble organic dye. In an embodiment, the film is a polarizing film. In an embodiment, the film is a phase-retarding film.

Another embodiment provides a liquid crystal display comprising at least one E-type polarizer. In an embodiment, the at least one E-type polarizer comprises at least one optically anisotropic film as described herein and a substrate. An embodiment provides a dichroic light-polarizing element comprising a substrate and at least one LLC film as described herein. In some embodiments, the dichroic light-polarizing element is an E-type polarizer. One embodiment provides a liquid crystal active display comprising at least one E-type polarizer film, wherein the E-type polarizer film comprises at least one LLC film as described herein. Conventional LC displays often use O-type films, and the contrast ratio can drop off drastically when the LC display is viewed from an angle off the normal directly. Conversely, a LC display comprising at least one E-type polarizer film may provide wide viewing angles without a substantial drop in contrast ratio. Furthermore, in preferred embodiments the process of making an E-type polarizer comprising an LLC film as described herein can be conducted more easily compared to the conventional process for making O-type polarizers. This also can lead to simplified and lower cost LC devices. The designs and components of a LC display comprising an E-type polarizer are described in more detail in U.S. Pat. No. 7,015,990, which is also incorporated by reference in its entirety, and particularly for the purpose of describing such designs and components.

Another embodiment provides a method of forming an optically anisotropic film. In an embodiment, the method of forming an optically anisotropic film comprises applying an LLC system as described herein onto a substrate, wherein the LLC system comprises a plurality of LLC mesophases, and orienting the plurality of LLC mesophases. In an embodiment, the method further comprises forming the LLC system by mixing at least one chromophoric compound described herein with water or a mixture of water and an organic solvent. In an embodiment, the method comprises drying the LLC system on the substrate. In an embodiment, the orienting of the plurality of LLC mesophases comprises spreading the LLC mesophases in one direction.

### EXAMPLES

### Example 1

### Synthesis

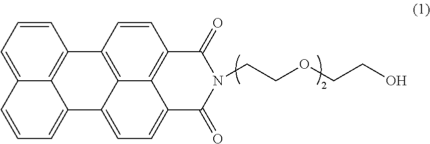

Step I-1. 2-[2-(2-aminoethoxy)ethoxy]ethanol (1.64 g, 11 mmol), perylenedicarboxylic mono anhydride (1.6 g, 5 mmol), and anhydrous trimethyl amine (20 mL) were mixed in 40 mL of anhydrous DMSO under argon in a 250 mL flask. After the reaction mixture (sealed) was stirred overnight (10-14 hours) at 150° C., the reaction solution was cooled to 80° C. and poured into 900 mL of 10% HCl (aq). The resultant solution was stirred at room temperature for an additional 4 hours. The precipitate was collected by filtration, washed with water (100 mL×3) and dried at 60° C. under vacuum for 4 hours. The compound N-(2-(2-(2-hydroxyethoxy)ethoxy)ethyl) perylenedicarboxylic imide (1) (1.94 g, 84%%) was obtained as a dark red solid with sufficient purity for the next step of synthesis. If desired, the product can be further purified by silica gel chromatography eluted by CHCl₃/MeOH (12:1/v:v) (R_{f}=0.51).

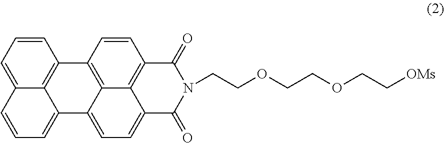

Step I-2. To a solution of N-(2-(2-(2-hydroxyethoxy)ethoxy)ethyl) perylenedicarboxylic imide (1) (1.8 g, 3.97 mmol) in 100 mL of anhydrous CHCl₃, anhydrous triethyl amine (2.3 mL, 1.68 g, 16.67 mmol) was added under Ar with stirring. After the solution was cooled to 0° C., methanesulfonyl chloride (1.3 mL, 1.91 g, 16.67 mmol) was added slowly by a syringe under Ar. Stirring was continued overnight at room temperature, followed by addition of 100 mL of CHCl₃. The mixture was washed with NaHCO₃ (5% w/w, 2×200 mL), H₂O (2×10 mL), and brine (100 mL). The organic phase was dried over MgSO₄, filtered and evaporated by rotary evaporator. The compound N-(2-(2-(2-methanesulfonylethoxy)ethoxy)ethyl)perylenedicarboxylic imide (2) (1.94 g, 92%) was obtained as a dark red solid. If desired, the product can be further purified by silica gel chromatography eluted by CHCl₃/MeOH (15:1/v:v) (R_{f}=0.52).

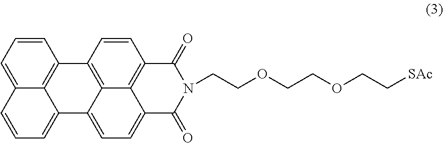

Step I-3. A mixture of N-(2-(2-(2-methanesulfonylethoxy)ethoxy)ethyl) perylenedicarboxylic imide (2) (1.8 g, 3.39 mmol) and potassium thioacetate (KSAc) (0.5 g, 4.38 mmol) in 25 mL anhydrous DMF was stirred at 50° C. for 24 hours during which the reaction flask was covered with aluminum foil. The reaction mixture was poured into water (250 mL) and extracted by CHCl₃ (3×300 mL). The combined organic phases were washed with water (100 mL), NaHCO₃ (aq) (5% w/w, 10 mL), and brine (100 mL). The organic phase was dried over MgSO₄, filtered, and evaporated by rotary evaporator. The result residue was purified by silica gel column (CHCl₃/MeOH=15:1/v:v) to produce N-(2-(2-(2-thioacetylethoxy)ethoxy)ethyl)perylenedicarboxylic imide (3) as a dark solid (1.42 g, 82%) (R_{f}=0.62).

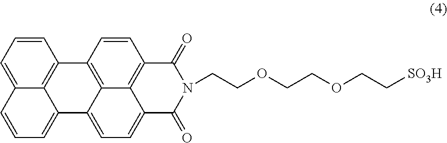

Step I-4. A mixture of H₂O₂ (30%, w/w, 6 mL) and acetic acid (20 mL) was added to a solution of N-(2-(2-(2-thioacetylethoxy)ethoxy)ethyl)perylenedicarboxylic imide (3) (1.4 g, 2.74 mmol) in 15 mL of acetic acid. After stirring for 24 hours, 10% Pd/C (40 mg) was added to destroy the excess hydrogen peroxide. The reaction mixture was filtered, concentrated, and co-evaporated with toluene (2×20 mL) and ether (2×20 mL) under reduced pressure (e.g., in a rotary evaporator) at 70° C. to yield the sulfonic acid derivative (4). The compound (4) was further purified by recrystallization from water/isopropanol to give the purified compound (4). The sulfonic acid derivative (4) (1.02 g, 72%) was obtained as dark red solid.

Step II-a-1. Synthesis of p-toluenesulfonic acid 2-[2-[2-(2-hydroxyethoxy)ethoxy]ethoxy]ethyl ester:

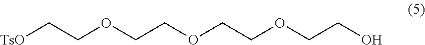

Tetra(ethylene glycol) (40 mL, 22 mmol) was added to a solution of p-toluenesulfonyl chloride (44 g, 24 mmol) and dimethylaminopyridine (DMAP) (36 g, 26 mmol) in 150 mL of anhydrous dichloromethane at 0° C. under argon. The reaction mixture was then stirred for 2 hours at 0° C., followed by continued stirring overnight at room temperature under argon. Detection of the product by thin layer chromotography (TLC) was accomplished using UV light, phosphomolybdic acid solution (10% PMA in EtOH), or iodine. After removal of the precipitate by filtration, the solution was evaporated under reduced pressure. The residue was purified by chromatography on a silica gel column eluted with EtOAc/Hexane (80:20 to 100:0/v:v), and compound (5) was obtained (35 g, 45% yield) as colorless oil. R_{f}=0.2 (EtOAc).

Step II-a-2. Synthesis of 2-[2-[2-(2-azidoethoxy)ethoxy]ethoxy]ethanol:

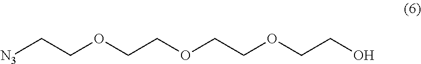

A solution of p-toluenesulfonic acid 2-[2-[2-(2-hydroxyethoxy)ethoxy]ethoxy]ethyl ester (5) (6 gram, 17.2 mmol) and sodium azide (1.7 gram, 26.2 mmol) in 50 mL of anhydrous MeCN was refluxed for 36 hours. After returning to room temperature, 50 mL of water was added and the mixture was extracted with CH₂Cl₂ Detection of the product on the TLC was accomplished using sulfuric acid solution (25 mL of conc. Sulfuric acid, 12.6 g of ammonium molybdate, 0.57 g of cerium, and 225 mL of deionized water) or iodine. The organic phase was then chromatographed on a silica gel column eluted with EtOAc. The compound (6) was obtained as a colorless oil (3.3 g, 88%). R_{f}=0.5 (EtOAc).

Step II-a-3. Synthesis of 2-[2-[2-(2-aminoethoxy)ethoxy]ethoxy]ethanol:

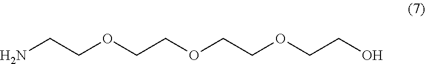

The azido product 2-[2-[2-(2-azidoethoxy)ethoxy]ethoxy]ethanol (6) (4.2 g, 19.2 mmol), triphenylphopshine (5.76 g, 22 mmol), and water (539 mg, 29.5 mmol) were mixed with 20 mL THF. After the solution was stirred for 4 hours at room temperature, the solvent was removed by rotary evaporator and the residue was purified on a silica gel column eluted with CHCl₃/MeOH/Et₃N (3:3:1). The compound (7) was obtained as colorless oil (3.3 g, 89% yield).

Step II-a-4. Synthesis of Bis-N,N-(2-(2-(2-(2-hydroxyethoxy)ethoxy)ethoxy)ethyl) perylenetetracarboxylic diimide:

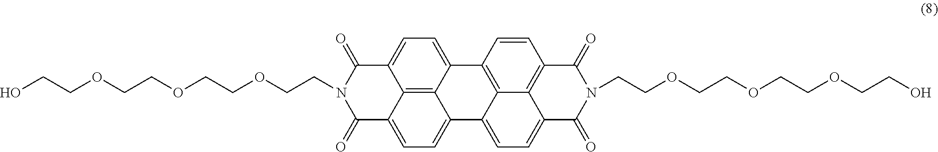

2-[2-[2-(2-aminoethoxy)ethoxy]ethoxy]ethanol (7) (2.6 g, 13.5 mmol), perylenetetracarboxylic dianhydride (2.2 g, 5.6 mmol) and anhydrous trimethyl amine (25 mL) were mixed in 50 mL of anhydrous DMSO under argon in a 250 mL flask. After the reaction mixture (sealed) was stirred overnight (10-14 hours) at 150° C., the reaction solution was cooled to 80° C. and poured into 900 mL of 10% HCl (aq). The resultant solution was stirred at room temperature for additional 4 hours. The precipitate was collected by filtration, washed with water (100 mL×3) and dried at 60° C. under vacuum for 4 hours. The compound (8) (4 g, 96%) was obtained as dark red solid, which is sufficient purity for the next step of synthesis. If desired, the product can be further purified by silica gel chromatography eluted by CHCl₃/MeOH (10:1/v:v)) (R_{f}=0.45).

Step II-a-5. Synthesis of Bis-N,N-(2-(2-(2-(2-methanesulfonylethoxy)ethoxy)ethoxy)ethyl)perylenetetracarboxylic diimide:

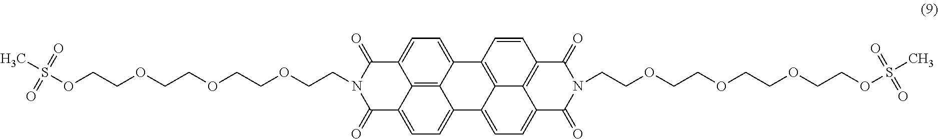

To a solution of bis-N,N-(2-(2-(2-(2-hydroxyethoxy)ethoxy)ethoxy)-ethyl) perylenetetracarboxylic diimide (8) (4.2 g, 5.66 mmol) in 150 mL of anhydrous CHCl₃, anhydrous triethyl amine (2.3 mL, 1.68 g, 16.67 mmol) was added under Ar with stirring. After the solution was cooled to 0° C., methanesulfonyl chloride (1.3 mL, 1.91 g, 16.67 mmol) was added slowly by a syringe under argon. Stirring was continued overnight at room temperature, followed by addition of 200 mL of CHCl₃. The mixture was washed with NaHCO₃ (5% w/w, 2×200 mL), H₂O (2×10 mL), and brine (100 mL). The organic phase was dried over MgSO₄ filtered and evaporated by rotary evaporator. The compound (9) (4.8 g, 94%) was obtained as dark red solid. If desired, the product can be further purified by silica gel chromatography eluted by CHCl₃/MeOH (10:1/v:v)) (R_{f}=0.55).

Step II-a-6: Synthesis of Bis-N,N-(2-(2-(2-(2-thioacetylethoxy)ethoxy)ethoxy)ethyl) perylenetetracarboxylic diimide:

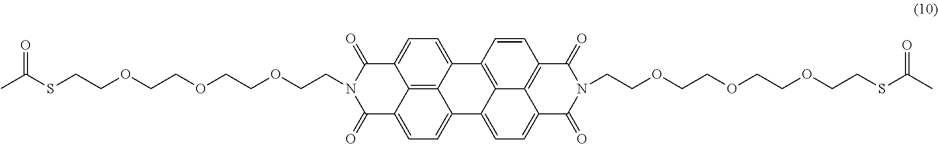

A mixture of Bis-N,N-(2-(2-(2-(2-methanesulfonylethoxy)ethoxy)ethoxy)ethyl) perylenetetracarboxylic diimide (9) (1.5 g, 1.67 mmol) and potassium thioacetate (KSAc) (0.5 g, 4.38 mmol) in 25 mL anhydrous DMF was stirred at 50° C. for 24 hours during which the reaction flask was covered with aluminum foil. The reaction mixture was poured into water (250 mL) and extracted by CHCl₃ (3×300 mL). The combined organic phases were washed with water (100 mL), NaHCO₃ (aq) (5% w/w, 10 mL), and brine (100 mL). The organic phase was dried over MgSO₄, filtered and evaporated by rotary evaporation. The resulting residue was purified by silica gel column (CHCl₃/MeOH=10:1/v:v) to produce (10) as a dark solid (1.21 g, 84%) (R_{f}=0.68).

Step II-a-7. Synthesis of bis-N,N-(2-(2-(2-(2-sulfonic acid ethoxy)ethoxy)ethyl) perylenetetracarboxylic diimide:

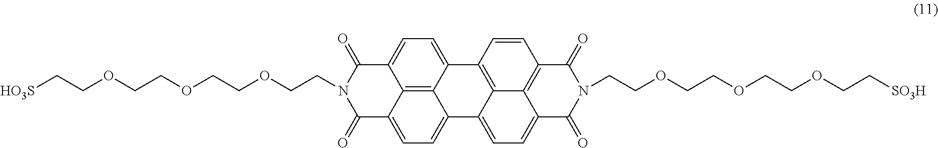

A mixture of H₂O₂ (30%, w/w, 5 mL) and acetic acid (25 mL) was added to a solution of Bis-N,N-(2-(2-(2-(2-thioacetylethoxy)ethoxy)ethoxy)ethyl)perylenetetracarboxylic diimide (10) (1.2 g, 1.4 mmol) in 15 mL of acetic acid. After stirring for 24 hours, 10% Pd/C (50 mg) was added to react with the excess hydrogen peroxide. The reaction mixture was filtered, concentrated, and co-evaporated with toluene (2×20 mL) and ether (2×20 mL) under reduced pressure (e.g., in a rotary evaporator) at 70° C. to yield sulfonic acid (11). The compound (11) was purified by recrystallization from water/isopropanol to give the purified compound (11). The sulfonic acid compound (11) (910 mg, 74%) was obtained as a dark red solid.

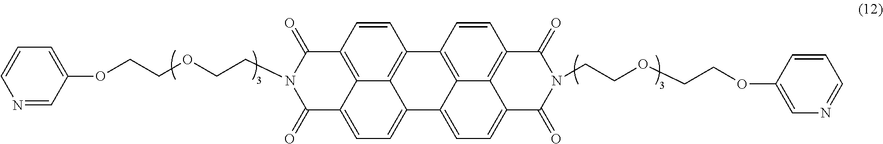

Step II-b-1. Bis-N,N-(2-(2-(2-(2-methanesulfonylethoxy)ethoxy)ethoxy)ethyl) perylenetetracarboxylic diimide (1.8 g. 2 mmol) was added to a solution of 3-hydroxylpyridine (570 mg, 6 mmol) and K₂CO₃ (1.38, 100 mmol)) in 20 mL of anhydrous DMF. The resultant mixture was heated to 80° C. with stirring under argon for 5 hours. After cooling to room temperature, the reaction mixture was treated with 200 mL of CHCl₃ and 150 mL of water. The organic phase was collected and dried over anhydrous Na₂SO₄. The organic solvent was removed by rotary evaporation to give the crude product bis-N,N-(2-(2-(2-(3-pyridyloxyethoxy)ethoxy)ethoxy)ethyl) perylenetetracarboxylic diimide (12). The product was purified by chromatography using CHCl₃/MeOH (12:1/v:v) to provide pure (12) (1.52, 85%).

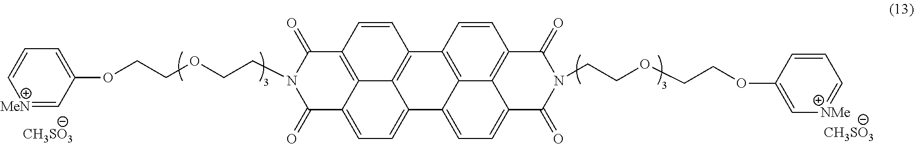

Step II-b-2. To a solution of Bis-N,N-(2-(2-(2-(3-pyridyloxyethoxy)ethoxy)ethoxy)ethyl) perylenetetracarboxylic diimide (12) (1.45 g, 1.62 mmol) in 10 mL of CHCl₃, 3 mL of CH₃SO₃Me (2.2 g, 20 mmol) was added. After the mixture was stirred for 24 hours, the product bis-N,N-(2-(2-(2-(3-pyridyloxyethoxy)ethoxy)ethoxy)ethyl)perylenetetracarboxylic diimide (13) precipitated, and the precipitate was washed with ether and methanol to give pure (13) (1.72 g 95%).

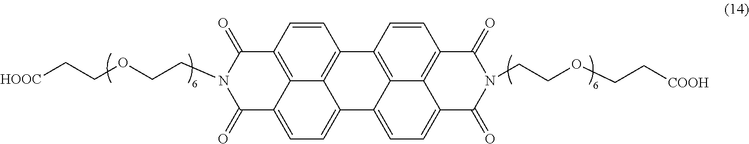

The perylenetetracarboxylic dianhydrides (1.96 g, 5 mmol)) and 3-[2-[2-[2-[2-[2-(2-aminoethoxy)ethoxy]ethoxy]ethoxy]ethoxy]ethoxy]propanoic acid (3.88 g, 11 mmol) were mixed in 20 mL of anhydrous DMSO. After sonicating for 10 minutes, the mixture was irradiated at 160° C. for 40 minutes using a microwave reactor and then cooled to room temperature. The solvent was distilled off under vacuum. The residue was purified by recrystallization from CHCl₃/hexane. The product bis-N,N-[2-[2-[2-[2-[2-(3-propanoic acid ethoxy)ethoxy]ethoxy]ethoxy]ethoxy]ethoxy]perylenetetracarboxylic diimide (14) can be further purified by silica gel chromatography eluted by CHCl₃/MeOH (4:1/v:v)), if desired (R_{f}=0.55) (4.3, 81%).

### Example 2

### Measurement of Dichroic Ratios

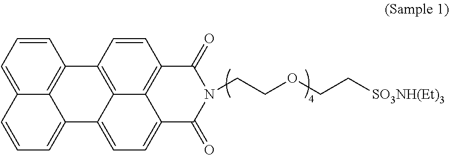

A 15 wt % solution of Sample 1 in deionized water was prepared by dissolving 150 mg of Sample 1 in 0.85 mL of deionized water. A standard glass slide was washed with 1% alcohol solution in an ultrasonic tank for 60 minutes and later rinsed with deionized water, isopropyl alcohol and dried in room temperature. The Sample 1 solution was coated onto the glass slide (2 inches by 3 inches by 1 mm), with a applicator rod (⅜ inch in diameter, #2½ wire size, Paul N. Gardner Co. Inc.) at a linear velocity of 25 mm/s. The resulting film thickness was approximately 0.2 micrometers. The coating process was conducted at room temperature (˜20° C.) and a relative humidity of about 65% and the film was dried under the same conditions.

The film was characterized by absorbance spectra measured on a Perkin Elmer Lamda Bio 40 UV/Vis Spectrum spectrophotometer in a wavelength range from 190 nm to 800 nm using a light beam polarized along the direction of the film application (Aₚₐᵣ) and in the perpendicular direction (Aₚₑᵣ) relative to the film application direction. At a wavelength of λ=420 nm corresponding to maximum absorption, the dichroic ratio K_{d}=log (Aₚₐᵣ)/log (Aₚₑᵣ) was equal to about 3.

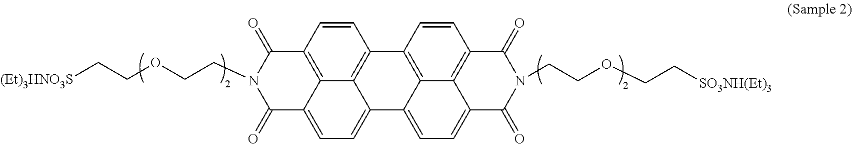

A 15 wt % solution of Sample 2 in deionized water was prepared by dissolving 150 mg of Sample 2 in 0.85 mL of deionized water. This solution was coated onto a standard glass slide by the same technique described for Sample 1. The resulting film thickness was approximately 0.2 μm.

The film was characterized by absorbance spectra measured on a Perkin Elmer Lamda Bio 40 UV/Vis Spectrum spectrophotometer in a wavelength range from 190 nm to 800 nm using a light beam polarized along the direction of the film application (Aₚₐᵣ) and in the perpendicular direction (Aₚₑᵣ) relative to the film application direction. At a wavelength of λ=485 nm corresponding to maximum absorption, the dichroic ratio K_{d} was equal to about 37.

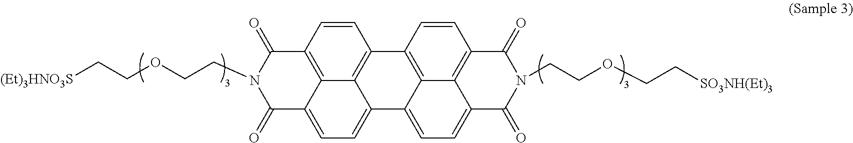

A 15 wt % solution of Sample 3 in deionized water was prepared by dissolving 150 mg of Sample 3 in 0.85 mL of deionized water. This solution was coated onto a standard glass slide by the same technique described for Sample 1. The resulting film thickness was approximately 0.2 μm.

The film was characterized by absorbance spectra measured on a spectrophotometer in a wavelength range from 190 nm to 800 nm using a light beam polarized along the direction of the film application (Aₚₐᵣ) and in the perpendicular direction (Aₚₑᵣ) relative to the film application direction. At a wavelength of λ=485 nm corresponding to maximum absorption, the dichroic ratio K_{d} was equal to about 11.

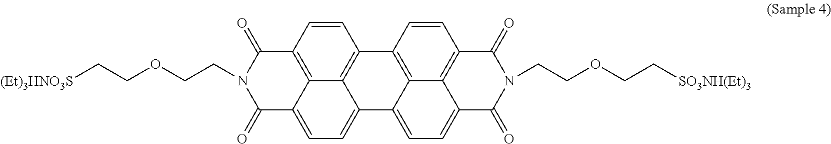

A 15 wt % solution of Sample 4 in deionized water was prepared by dissolving 150 mg of Sample 4 in 0.85 mL of deionized water. This solution was coated onto a standard glass slide by the same technique described for Sample 1. The resulting film thickness was approximately 0.2 p.m.

The film was characterized by absorbance spectra measured on a spectrophotometer in a wavelength range from 190 nm to 800 nm using a light beam polarized along the direction of the film application (Aₚₐᵣ) and in the perpendicular direction (Aₚₑᵣ) relative to the film application direction. At a wavelength of λ=485 nm corresponding to maximum absorption, the dichroic ratio K_{d} was equal to about 28.

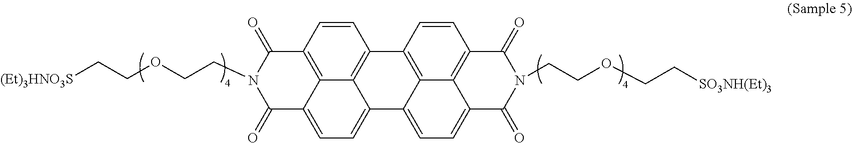

A 15 wt % solution of Sample 5 in deionized water was prepared by dissolving 150 mg of Sample 5 in 0.85 mL of deionized water. This solution was coated onto a standard glass slide by the same technique described for sample 1. The resulting film thickness was approximately 0.2 μm.

The film was characterized by absorbance spectra measured on a spectrophotometer in a wavelength range from 190 nm to 800 nm using a light beam polarized along the direction of the film application (Aₚₐᵣ) and in the perpendicular direction (Aₚₑᵣ) relative to the film application direction. At a wavelength of λ=485 nm, which is corresponding to maximum absorption, the dichroic ratio Kd was equal to about 31.

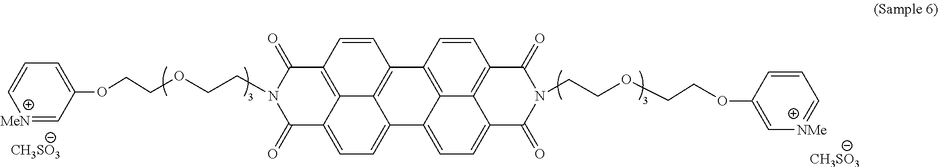

A 15 wt % solution of Sample 6 in deionized water was prepared by dissolving 150 mg of Sample 6 in 0.85 mL of deionized water. This solution was coated onto a standard glass slide by the same technique described for sample 1. The resulting film thickness was approximately 0.2 μm.

The film was characterized by absorbance spectra measured on a spectrophotometer in a wavelength range from 190 nm to 800 nm using a light beam polarized along the direction of the film application (Aₚₐᵣ) and in the perpendicular direction (Aₚₑᵣ) relative to the film application direction. At a wavelength of λ=485 nm, which is corresponding to maximum absorption, the dichroic ratio Kd was equal to about 9.

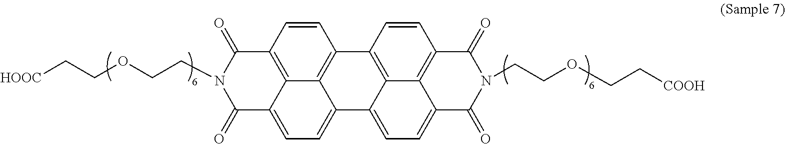

A 15 wt % solution of Sample 7 in deionized water was prepared by dissolving 150 mg of Sample 7 in 0.85 mL of deionized water. This solution was coated onto a standard glass slide by the same technique described for sample 1. The resulting film thickness was approximately 0.2 μm.

The film was characterized by absorbance spectra measured on a spectrophotometer in a wavelength range from 190 nm to 800 nm using a light beam polarized along the direction of the film application (Aₚₐᵣ) and in the perpendicular direction (Aₚₑᵣ) relative to the film application direction. At a wavelength of λ=485 nm, which is corresponding to maximum absorption, the dichroic ratio Kd was equal to about 7.

### Comparative Example 1

Several compounds described in Japanese Pat. App. 2006-098927 were synthesized. The comparative compounds were as follows:

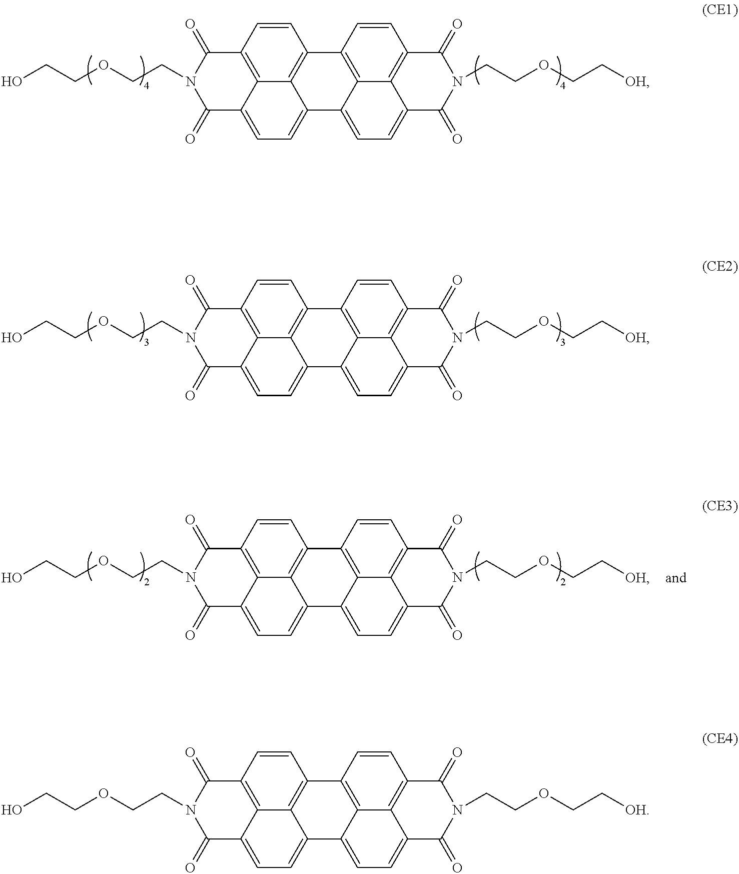

The solubility and K_{d} of each of these compounds were measured in the manner described above. The solubility of compound CE1 in water is about 10% by weight and the K_{d} is less than about 9. The solubility of compound CE2 in water is less than about 0.1% by weight and the K_{d} is less than about 9. Each of the compounds CE3 and CE4 have a solubility in water of less than about 0.1% by weight and a K_{d} of less than about 7.

The above description discloses several methods and materials of the preferred embodiments. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention as embodied in the attached claims.

What is claimed is:

1. A lyotropic chromophoric compound having the general structural formula (I), the general structural formula (II), or the general structural formula (III): 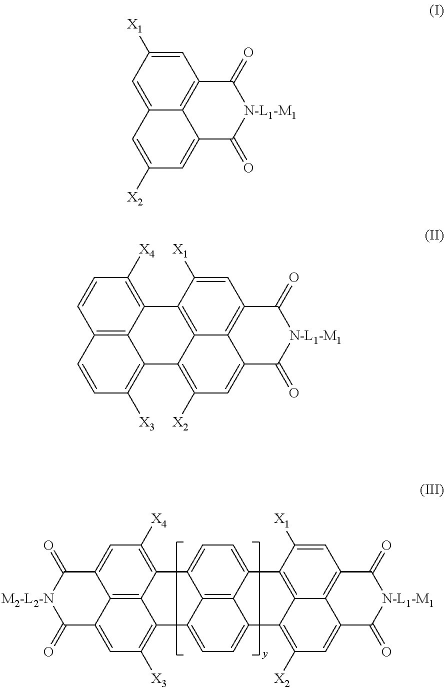
wherein L₁ and L₂ each independently represent a hydrophilic linker; M₁ and M₂ each independently represent an acidic group, a basic group, or salt thereof; X₁, X₂, X₃ and X₄ are each independently selected from —H, —NHCH₃, a pyrrolidinyl group, or a halogen; and y is an integer in the range from 0 to about 4.

2. The compound of claim 1, wherein L₁ and L₂ are each independently selected from a linker having the general formula (IV), a linker having the general formula (V) and a linker having the general formula (VI): 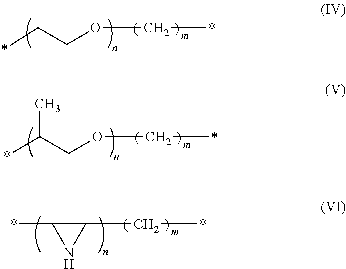
wherein each n is independently selected from an integer in the range of 1 to about 9 and each m is independently selected from an integer in the range of 0 to about 6.

3. The compound of claim 1, wherein M₁ and M₂ are each independently selected to comprise an anion portion independently selected from —SO₃⁻ and —CO₂⁻.

4. The compound of claim 1, wherein M₁ and M₂ are each independently selected to comprise a cation portion selected from: 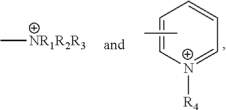
wherein R₁, R₂, R₃, and R₄ are each independently selected from hydrogen, an optionally substituted C₁ to C₆ alkyl group, an optionally substituted C₂ to C₆ alkenyl group, an optionally substituted C₂ to C₆ alkynyl group, an optionally substituted C₃ to C₈ cycloalkyl group, an optionally substituted aryl group, or an optionally substituted aralkyl group.

5. The compound of claim 4, wherein R₁, R₂, R₃, and R₄ are each independently selected from hydrogen, methyl, ethyl, n-propyl, isopropyl, n-butyl, isobutyl, sec-butyl, tert-butyl, and cyclohexyl.

6. The compound of claim 1, wherein each M₁ and M₂ further comprise a counter ion.

7. The compound of claim 6, wherein the counter ion is independently selected from H⁺, NH₄⁺, NH(Et)₃⁺, K⁺, Li⁺, Na⁺, Cs⁺, Ca⁺⁺, Sr⁺⁺, Mg⁺⁺, Ba⁺⁺, Co⁺⁺, Mn⁺⁺, Zn⁺⁺, Cu⁺⁺, Pb⁺⁺, Fe⁺⁺, Ni⁺⁺, Al³⁺, Ce³⁺, and La³⁺.

8. The compound of claim 6, wherein the counter ion is independently selected from CO₂CF₃⁻, CH₃SO₃⁻, Cl⁻, Br⁻, and I⁻.

9. The compound of claim 1, wherein the acidic group, basic group, or salt thereof comprises nitrogen.

10. The compound of claim 6, wherein one or more counter ions are shared by at least two molecules.

11. The compound of claim 2, wherein X₁, X₂, X₃ and X₄ are each —H, y is selected from the range of 0 to 2, n is an integer in the range of 1 to 4, and m is an integer in the range of 1 to 3.

12. The compound of claim 1, which is configured for π-π stacking.

13. A lyotropic liquid crystal system comprising at least one lyotropic chromophoric compound of claim 1.

14. The lyotropic liquid crystal system of claim 13, wherein the lyotropic liquid crystal system is water-based.

15. The lyotropic liquid crystal system of claim 13, wherein the lyotropic liquid crystal system comprises a mixture of water and an organic solvent miscible with water.

16. The lyotropic liquid crystal system of claim 13, wherein the concentration of the lyotropic chromophoric compound in the lyotropic liquid crystal system is in the range of about 5% to about 50% by weight of the lyotropic liquid crystal system.

17. The lyotropic liquid crystal system of claim 13, further comprising one or more surfactants in an amount of up to about 5% by weight of the lyotropic liquid crystal system.

18. The lyotropic liquid crystal system of claim 13, further comprising one or more plasticizers in an amount of up to about 5% by weight of the lyotropic liquid crystal system.

19. The lyotropic liquid crystal system of claim 13, comprising a combination of two or more lyotropic chromophoric compounds of the general structural formulae (I), (II), and/or (III), wherein the amount of compound according to formula (I) is in the range of about 0% to about 99% by weight, based on the total amount of chromophoric compounds, the amount of compound according to formula (II) is in the range of about 0% to about 99% by weight, based on the total amount of chromophoric compounds, and the amount of compound according to formula (III) is in the range of about 0% to about 99% by weight, based on the total amount of chromophoric compounds, provided that the total amount of compounds according to formulae (I), (II), and/or (III) accounts for at least 50% of the total weight of all the chromophoric compounds in the lyotropic liquid crystal system.

20. The lyotropic liquid crystal system of claim 13, further comprising at least one water-soluble organic dye or an organic compound, the organic dye or organic compound being configured to participate in the formation of a liquid crystal.

21. The lyotropic liquid crystal system of claim 13, wherein the lyotropic liquid crystal system comprises a lyotropic liquid crystal mesophase.

22. An optically anisotropic film comprising at least one lyotropic chromophoric compound of claim 1.

23. The optically anisotropic film of claim 22, wherein the film is formed by depositing a lyotropic liquid crystal system comprising at least one lyotropic chromophoric compound onto a substrate.

24. The optically anisotropic film of claim 22, wherein the film is at least partially crystalline.

25. The optically anisotropic film of claim 22, further comprising at least one water soluble organic dye.

26. The optically anisotropic film of claim 22, wherein the film is a polarizing film.

27. The optically anisotropic film of claim 22, wherein the film is a phase-retarding film.

28. A liquid crystal display comprising at least one E-type polarizer, wherein the at least one E-type polarizer comprises at least one optically anisotropic film of claim 22 and a substrate.

29. A method of forming an optically anisotropic film, comprising:
applying a lyotropic liquid crystal system of claim 13 on a substrate, wherein the lyotropic liquid crystal system comprises a plurality of liquid crystal mesophases; and
orienting the plurality of liquid crystal mesophases.

30. The method of claim 29, further comprising forming the lyotropic liquid crystal system by mixing at least one chromophoric compound of claim 1 with water or a mixture of water and an organic solvent.

31. The method of claim 29, further comprising drying said lyotropic liquid crystal system on the substrate.

32. The method of claim 29, wherein orienting the plurality of liquid crystal mesophases comprises spreading the lyotropic liquid crystal mesophases in one direction.
